(19) United States
(12) Patent Application Publication
(10) Pub. No.: US 2016/0081631 A1
(43) Pub. Date: Mar. 24, 2016

(54) RESPIRATORY WAVEFORM RECOGNITION METHOD AND SYSTEM

(71) Applicant: APEX MEDICAL CORP., New Taipei City (TW)

(72) Inventors: Kang-Ping LIN, New Taipei City (TW); Geng-Hong LIN, New Taipei City (TW); Hao-Yu JAN, New Taipei City (TW); Sheng-Cheng HUANG, New Taipei City (TW); Po-Chung SHIN, New Taipei City (TW); Ching-Liang YU, New Taipei City (TW); Da-Long LEE, New Taipei City (TW)

(21) Appl. No.: 14/493,888

(22) Filed: Sep. 23, 2014

(51) Int. Cl. A61B 5/00; A61B 5/08; A61B 5/087

(52) U.S. Cl. CPC A61B 5/7282; A61B 5/087; A61B 5/0826; A61B 5/7246; A61B 5/742

(57) ABSTRACT

A respiratory waveform recognition method comprises: (a) detecting a respiratory airflow in a respiratory cycle; (b) measuring an amplitude of the respiratory airflow and a duration of the respiratory cycle; (c) using a plurality of sampling points to determine an inspiration waveform and an expiration waveform according to the amplitude and the duration; (d) normalizing the amplitude and the duration of one of the inspiration waveform and the expiration waveform, so as to establish a normalized waveform; and (e) accumulating the differences between the normalized waveform and a reference waveform to calculate a flow index useful for the identification of a normal respiration state and an abnormal respiration state. A curve, such as a weighted curve or a standard waveform, is used for fitting of the inspiration waveform or the expiration waveform to calculate the differences, and the differences are accumulated to identify the normal respiration state and the abnormal respiration state. Also provided is a respiratory waveform recognition system.

### FIELD OF THE DISCLOSURE

The present disclosure relates to respiration state recognition and more particularly to a respiratory waveform recognition method and a system thereof, in which at least a portion of a respiratory airflow is fit to at least one of a curve, a weighted curve and a standard waveform.

### BACKGROUND

Conventionally, when a patient fails to acquire sufficient oxygen concentration during respiration (hereinafter referred to as abnormal respiration state), he or she may use a respiration machine to maintain the necessary respiration activity to ensure sufficient oxygen concentration supply.

The abnormal respiration state usually occurs when the patient is sleeping and may generally be categorized into apnea, hypopnea or flow limitation, as described in detail below.

Apnea refers to decreased blood oxygen in patients due to the cessation of airflow supply to lung for more than 10 seconds during sleep. Patients diagnosed with sleep apnea suffer from abnormal life and increased risk of cardiovascular diseases, heart attack and cerebral haemorrhage due to poor sleep quality.

Hypopnea refers to reduced airflow due to overly slow or shallow breathing during sleep. In addition, it may be accompanied by oxygen desaturation and arousal for tens of seconds. For example, if during sleep the respiration activity and airflow are reduced by 50% to 70% and the blood oxygen saturation is reduced by 4%, hypopnea can be properly diagnosed.

Flow limitation refers to reduced airflow passage due to partially obstructed airway. Specifically, events of repeated partial or complete obstruction of the upper airway may occur during sleep and result in temporary arousal of the patient because of dysfunctional upper airway muscles, thickening of larynx soft tissues, tonsil proliferation or tonsil hypertrophy.

Apnea-hypopnea index (AHI), which is the total number of apneas and hypopneas per hour, may be used for determining hypopnea or flow limitation. For an AHI greater than or equal to 15 or 5, the patient may have the problems associated with daytime sleepiness, snoring, witnessed apnea and arousal due to temporary choking and gasping.

Generally, snoring refers to the symptom where annoying sound is caused during sleep by the vibration of the soft tissues in throat, velum or uvula when airflow passes through narrowed upper airway.

The respiration machines mentioned above has been recognized as the most effective way to treat abnormal respiration state, wherein patients may receive continuous gas supply from the respiration machines to increase blood oxygen concentration.

Respiration machines can be categorized into positive pressure respiration machines and negative pressure respiration machines according to the style of pressure supply.

For positive pressure respiration machines, a respiratory airflow of patients is detected as the basis for assisting patients to perform respiration activity at a proper time.

Implantable detection technique and non-implantable detection technique are currently employed to detect a respiratory airflow. A detector is implanted into human body, such as heart ventricle, lung, or larynx, as the implantable detection technique, and an external positive pressure respiration machine is used for detecting the airflow as the non-implantable detection technique. The non-implantable detection technique does not require implantation and therefore allows quicker and easier detection of respiratory airflow of the patient without causing much discomfort than the implantable counterpart.

Even though the non-implantable detection technique may enable simple detection, there are still some drawbacks in the conventional respiration machines because these machines fail to effectively and precisely identify the severity of the abnormal respiration state and may undesirably inhibit patient's respiration when continuously providing pressure to the patient, which may worsen the respiration disorder.

Therefore, there is an unsolved need to provide a respiration machine allowing precise identification of severity and providing proper air pressure appropriately.

Accordingly, this disclosure provides a respiratory waveform recognition method and a system thereof capable of precisely identifying a normal respiration state and an abnormal respiration state from a respiratory airflow.

### SUMMARY

A first objective of the present disclosure is to provide a respiratory waveform recognition method, in which an amplitude and a duration of a respiratory airflow are timely normalized to provide a normalized respiratory airflow useful for an algorithm to precisely calculate a flow index useful for the identification of a normal respiration state and a abnormal respiration state, wherein the abnormal respiration state includes apnea, hypopnea and flow limitation.

A second objective of the present disclosure is to provide a respiratory waveform recognition method which captures an inspiration waveform or an expiration waveform from the respiratory airflow to reduce the computation load and time of the algorithm, wherein the respiratory waveform recognition method may identify the state of the respiratory airflow by any one of the inspiration waveform and the expiration waveform.

A third objective of the present disclosure is to provide a respiratory waveform recognition method, in which a curve fitting method is used for fitting the normalized inspiration waveform or expiration waveform to a first-order linear curve, a high-order linear curve, a weighted curve or a standard waveform, so as to determine the severity of the abnormal respiration state.

A fourth objective of the present disclosure is to provide a respiratory waveform recognition method, in which several sampling points in the respiratory airflow are redistributed through weighting during the fitting process, so as to promote the identification of the severity of the abnormal respiration state.

A fifth objective of the present disclosure is to provide a respiratory waveform recognition method, in which one of a plurality of standard waveforms most similar to the normalized inspiration waveform or the normalized expiration waveform is selected to calculate the errors between the two waveforms to determine the severity of the abnormal respiration state.

A sixth objective of the present disclosure is to provide a respiratory waveform recognition method, in which a portion of the normalized inspiration waveform or the normalized expiration waveform is selected, and the differences, such as the standard deviation of an absolute error, between the portion of the waveform and one of the standard waveforms are calculated to determine the severity of the abnormal respiration state.

A seventh objective of the present disclosure is to provide a respiratory waveform recognition system for detecting a respiratory airflow and identifying a normal respiration state and an abnormal respiration state therefrom.

In order to achieve these and other objectives, the present disclosure provides a respiratory waveform recognition method for identification of a normal respiration state and an abnormal respiration state from a respiratory airflow, the respiratory waveform recognition method comprising: (a) detecting the respiratory airflow in a respiratory cycle; (b) measuring an amplitude of the respiratory airflow and a duration of the respiratory cycle, wherein the duration is sufficient for detecting an ascending waveform or a descending waveform of the respiratory airflow; (c) using a plurality of sampling points to determine an inspiration waveform and an expiration waveform according to the amplitude and the duration; (d) normalizing the amplitude and the duration of one of the inspiration waveform and the expiration waveform, so as to establish a normalized waveform; and (e) accumulating the differences between the normalized waveform and a reference waveform to calculate a flow index useful for the identification of the normal respiration state and the abnormal respiration state.

In order to achieve these and other objectives, the present disclosure provides a respiratory waveform recognition method for identification of a normal respiration state and an abnormal respiration state from a respiratory airflow, the respiratory waveform recognition method comprising: (a1) detecting the respiratory airflow in a respiratory cycle; (b1) measuring an amplitude of the respiratory airflow and a duration of the respiratory cycle, wherein the duration is sufficient for detecting an ascending waveform or a descending waveform of the respiratory airflow; (c1) using a plurality of sampling points to determine an inspiration waveform and an expiration waveform according to the amplitude and the duration; (d1) normalizing the amplitude and the duration of one of the inspiration waveform and the expiration waveform, so as to establish a normalized waveform; (e1) fitting the normalized waveform to a linear equation; and (f1) using a weighted least-squares method to calculate the differences between the normalized waveform and the linear equation, and calculating a flow index from the differences with a weighting function, the flow index being useful for the identification of the normal respiration state and the abnormal respiration state.

In order to achieve these and other objectives, the present disclosure provides a respiratory waveform recognition method for identification of a normal respiration state and an abnormal respiration state from a respiratory airflow, the respiratory waveform recognition method comprising: (a2) detecting the respiratory airflow in a respiratory cycle; (b2) measuring an amplitude of the respiratory airflow and a duration of the respiratory cycle, wherein the duration is sufficient for detecting an ascending waveform or a descending waveform of the respiratory airflow; (c2) using a plurality of sampling points to determine an inspiration waveform and an expiration waveform according to the amplitude and the duration; (d2) normalizing the amplitude and the duration of one of the inspiration waveform and the expiration waveform, so as to establish a normalized waveform; (e2) comparing a plurality of standard waveforms and the normalized waveform, so as to set one of the standard waveforms as a reference waveform which is most similar to the normalized waveform among the standard waveforms; and (f2) accumulating the differences between the normalized waveform and a reference waveform to calculate a flow index useful for the identification of the normal respiration state and the abnormal respiration state.

In order to achieve these and other objectives, the present disclosure provides a respiratory waveform recognition system for identification of a normal respiration state and an abnormal respiration state from a respiratory airflow, the respiratory waveform recognition system comprising an airflow sensor, a processing unit and a display unit. The airflow sensor detects flow variation of the respiratory airflow for a duration, wherein the duration is sufficient for detecting an ascending waveform or a descending waveform of the respiratory airflow. The processing unit is connected to the airflow sensor for receiving a signal corresponding to the flow variation of the respiratory airflow, using an algorithm to analyze an amplitude of the respiratory airflow and measuring a duration of a respiratory cycle to calculate a flow index, the algorithm being adapted to determine, according to the flow index, whether the respiratory airflow represents the normal respiration state or the abnormal respiration state. The display unit is connected to the processing unit for displaying the normal respiration state or the abnormal respiration state. The algorithm samples the amplitude and the duration of a plurality of sampling points, the amplitude and the duration determine an inspiration waveform and an expiration waveform, and the algorithm normalizes the amplitude and the duration of one of the inspiration waveform and the expiration waveform, so as to establish a normalized waveform. The algorithm is configured for obtaining the flow index by calculating the differences between a reference waveform and the normalized waveform, or by calculating the differences between a linear equation and the normalized waveform.

In brief, the respiratory waveform recognition method and system of the present disclosure may fit the waveform of a respiratory airflow with at least one of multiple curves and calculate the differences therebetween. A flow index is calculated according to the differences, and the flow index may determine whether the respiratory airflow represents the normal respiration state or the abnormal respiration state. In addition to the determination of different respiration states, the flow index may further identify the severity of the abnormal respiration state, such as apnea, hypopnea, or flow limitation.

Notably, for hypopnea or flow limitation, the method and system of the present disclosure may further derive an AHI index from the flow index so as to determine obstructive sleep apnea or snoring symptom.

The present disclosure solves the problems of conventional respiration machines which fail to effectively determine the severity of the abnormal respiration state and undesirably inhibit patient's respiration when continuously providing pressure to the patient, which may worsen the respiration disorder.

### BRIEF DESCRIPTION OF THE DRAWINGS

Many aspects of the present embodiments can be better understood with reference to the following drawings. Moreover, in the drawings, like reference numerals designate corresponding parts throughout the several views.

FIG. 1 illustrates a flowchart of one embodiment of the respiratory waveform recognition method according to the present disclosure;

FIG. 2(a) illustrates a waveform representing a normal respiration state;

FIG. 2(b) illustrates a waveform representing an abnormal respiration state;

FIG. 3 illustrates a waveform representing the abnormal respiration state of FIG. 2(b) after amplitude normalization and duration or time normalization;

FIG. 4 illustrates several standard waveforms S(i); and

FIG. 5 illustrates a block diagram of one embodiment of the respiratory waveform recognition system according to the present disclosure.

### DETAILED DESCRIPTION

Embodiments of the present disclosure will now be described in detail below and with reference to the drawings.

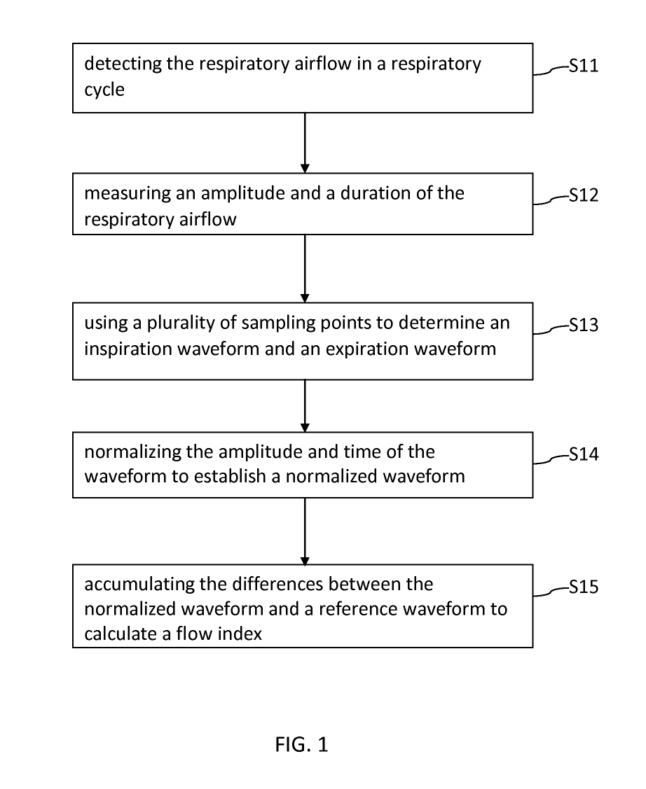

FIG. 1 illustrates a flowchart of one embodiment of the respiratory waveform recognition method according to the present disclosure, the respiratory waveform recognition method being capable of identifying and/or determining a normal respiration state and/or an abnormal respiration state from the waveform of a respiratory airflow.

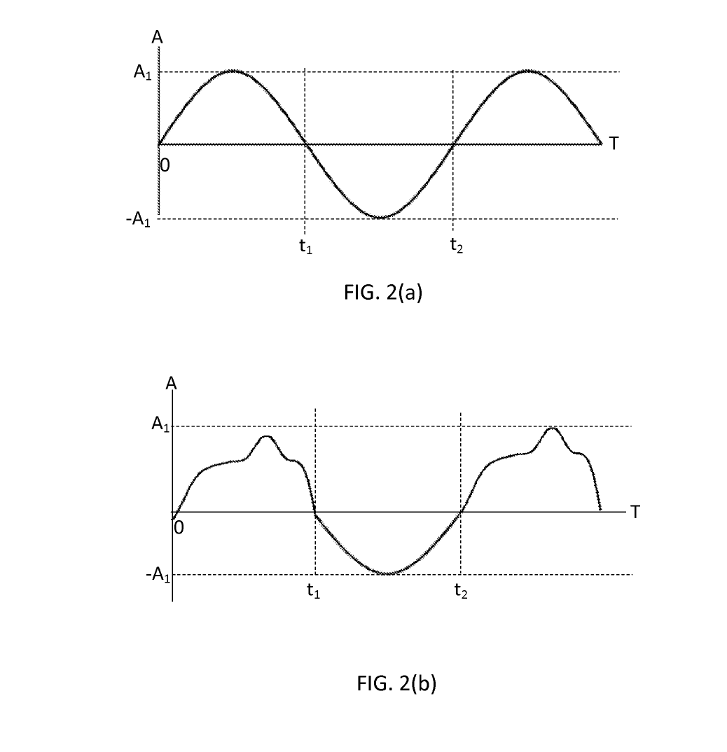

FIG. 2(a) illustrates a waveform representing a normal respiration state. FIG. 2(a) may also represent the waveform reflecting the variation of airflow in the airway of a patient during inspiration and expiration, wherein the x-axis represents time T and the y-axis represents amplitude A. The waveform between time 0 and t₁ may represent the inspiration airflow variation, and the waveform between time t₁ and t₂ may represent expiration airflow variation. In the normal respiration state, the inspiration airflow variation is substantially equal to the expiration airflow variation.

FIG. 2(b) illustrates a waveform representing an abnormal respiration state. In the abnormal respiration state, the airway is influenced by different obstruction degrees, so the inspiration airflow variation is not equal to the expiration airflow variation.

Referring back to FIG. 1, the respiratory waveform recognition method starts from step S11 for detecting the respiratory airflow in a respiratory cycle. Taking FIG. 2(b) as the example, the respiratory cycle is defined as the duration of completing one inspiration and one expiration, i.e. the cycle from time 0 to t₂.

Step S12 is adapted for measuring an amplitude of the respiratory airflow and a duration of the respiratory cycle. In FIG. 2(b), the amplitude represents the magnitude between A₁ to −A₁, and the duration represents the span between time 0 to t₂.

Step S13 is adapted for using a plurality of sampling points to determine an inspiration waveform (from time 0 to t₁) and an expiration waveform (from time t₁ to t₂) according to the amplitude A and the duration T. For example, sampling at a low frequency, such as several Hz, may be employed to create corresponding sampling points on the coordinate of amplitude A and corresponding time T, so as to delineate the waveform.

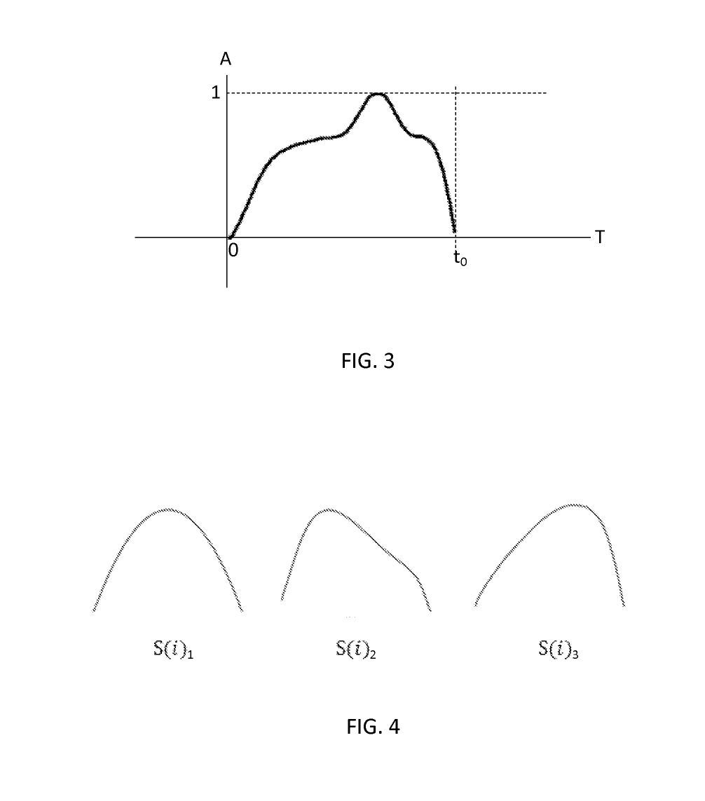

Step S14 is adapted for normalizing the amplitude A and the time T of one of the inspiration waveform and the expiration waveform, so as to establish a normalized waveform, as shown in FIG. 3, which represents a normalized inspiration waveform. In FIG. 3, because the amplitude of the inspiration waveform has been normalized, the magnitude of the amplitude A is normalized from A₁ to 1, and the length of time T is normalized from t₁ to t₀.

Step S15 is adapted for accumulating the differences, such as the least absolute error value and/or the least square error value, between the normalized waveform and a reference waveform to provide a flow index for determining whether the normalized waveform is identified as the normal respiration state or the abnormal respiration state. Several different types of reference waveforms may be used, as described below.

a) First-Order Linear Equation

The reference waveform may be in compliance with a first-order linear equation α₁·i+α₂, wherein the flow index has the mathematical representation Σᵢ₌ₓ^{y}|C1(i)−F(i)|, wherein C1(i) is the first-order linear equation, α₁ and α₂ are constants, x is a starting point of the sampling points, y is an ending point of the sampling points, i is each sampling point between x and y, and F(i) is the amplitude A of the normalized waveform at the sampling point i.

b) Second-Order Linear Equation

The reference waveform may be in compliance with a second-order linear equation α₁·i²+α₂·i+α₃, wherein the flow index has the mathematical representation Σᵢ₌ₓ^{y}|C2(i)−F(i)|, wherein C2(i) is the second-order linear equation, α₁, α₂ and α₃ are constants, x is a starting point of the sampling points, y is an ending point of the sampling points, i is each sampling point between x and y, and F(i) is the amplitude A of the normalized waveform at the sampling point i.

Because the second-order linear equation is plotted as a curve, it provides better fitting than the first-order linear equation.

c) Third-Order Linear Equation

The reference waveform may be in compliance with a third-order linear equation α₁·i³+α₂·i²+α₃·i+α₄, wherein the flow index has the mathematical representation Σᵢ₌ₓ^{y}|C3(i)−F(i)|, wherein C3(i) is the third-order linear equation, α₁, α₂, α₃ and α₄ are constants, x is a starting point of the sampling points, y is an ending point of the sampling points, i is each sampling point between x and y, and F(i) is the amplitude A of the normalized waveform at the sampling point i.

Compared with the fitting effect of the second-order linear equation, the third-order linear equation could further provide the capability of reflecting left or right shifting of the curve.

d) Third-Order Weighted Linear Equation

The reference waveform may be in compliance with a third-order weighted linear equation α₁·i³+α₂·i²+α₃·i+α₄, wherein the flow index has the mathematical representation Σᵢ₌ₓ^{y}W(i)×|C4(i)−F(i)|, wherein W(i) is defined below:

$$W ( i ) \{ \begin{matrix}A , & i = x , y \\ B , & i = MAX ( F ( i ) ) \\ C , & others\end{matrix} ;$$

wherein C4(i) is the third-order linear equation, α₁, α₂, α₃ and α₄ are constants, x is a starting point of the sampling points, y is an ending point of the sampling points, i is each sampling point between x and y, W(i) is a weighting function, A, B and C are weighting factors, and F(i) is the amplitude A of the normalized waveform at the sampling point i.

Compared with the fitting effect of the third-order linear equation, the third-order weighted linear equation could further weight for example the starting point x or the ending point y so as to adjust the end points of the curve delineated by the third-order weighted linear equation and reduce the error caused by the end points.

In this embodiment, the third-order weighted linear equation cites only the starting point and the ending point as the example, but this is for illustrative purpose only and not for limiting. In other words, other sampling points may also be weighted in addition to or instead of the starting point and the ending point.

In one embodiment, the weighting factor A ranges from 50 to 100, the weighting factor B ranges from 200 to 400, and the weighting factor C is 1.

e) Standard Waveform

The reference waveform may also be in compliance with one of a plurality of standard waveforms S(i), as illustrated in FIG. 4, in which waveforms S(i)₁, S(i)₂ and S(i)₃ of three normal respiration states are exemplified. The flow index has the following mathematical representation:

$$\sqrt{\frac{{\sum }_{i = x}^{y} {( Ki - μ )}^{2}}{L}} ;$$

$$wherein$$

$$Ki = {\sum }_{i = x}^{y} R \times \vert F ( i ) - S ( i ) \vert ;$$

$$μ = \frac{1}{L} \sum Ki ;$$

$$R = \frac{\sum S ( i )}{\sum F ( i )} ;$$

wherein x is a starting point of the sampling points, y is an ending point of the sampling points, i is each sampling point between x and y, L is the duration between the starting point and the ending point, Ki is an absolute error between any one of the standard waveforms and the normalized waveform, μ is the mean of absolute errors, R is a ratio of area under curve of any one of the standard waveforms and the normalized waveform, and F(i) is the amplitude A of the normalized waveform at the sampling point i.

In one embodiment, the starting point is set to 20% of the sampling points and the ending point is set to 80% of the sampling points.

In various embodiments of the reference waveforms a) to e), a plurality of flow thresholds can be set for the flow index. By comparing the flow index with the flow thresholds, the airway obstruction degree can be further determined from the flow index, such as clear airway, mild obstruction, severe obstruction or snoring.

Notably, although first-order to third-order linear equations are cited as the examples for the reference waveform, the reference waveform of the present disclosure is not limited thereto and can be expanded to other higher-order linear equations. For example, other higher-order linear equations can be plotted as curve waveforms for fitting the normalized waveform, and then the differences between the two waveforms are calculated to obtain the flow index for identifying the normalized waveform as a normal respiration state or an abnormal respiration state. Similarly, a weighting function may be introduced to other higher-order linear equations to reduce the error from the end points.

Notably, to achieve more precise recognition, identification or determination, the flow index calculated from the higher-order linear equations may also be compared with the flow thresholds to identify the degree of obstruction in the airway.

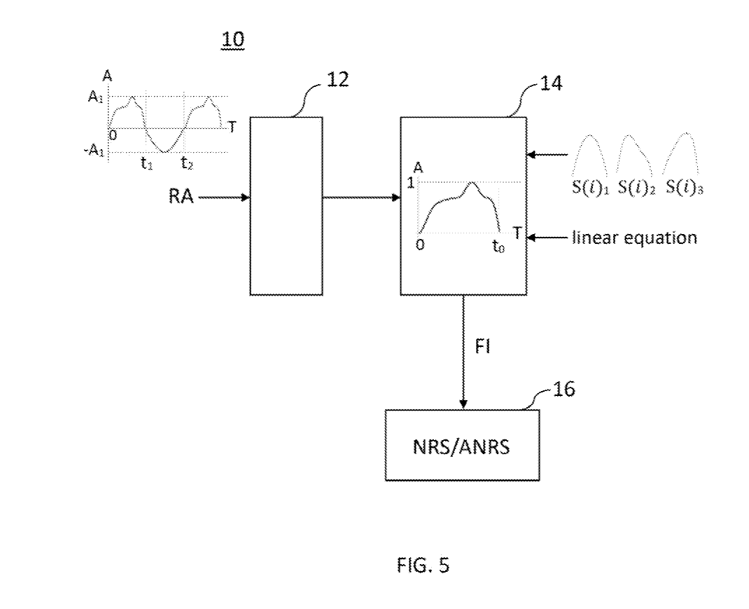

FIG. 5 illustrates a block diagram of one embodiment of the respiratory waveform recognition system according to the present disclosure. The respiratory waveform recognition system 10 is capable of identifying a normal respiration state NRS and an abnormal respiration state ANRS from a respiratory airflow RA.

The respiratory waveform recognition system 10 comprises an airflow sensor 12, a processing unit 14 and a display unit 16.

In a period of time, the airflow sensor 12 detects the flow variation of the respiratory airflow RA, and the length of the period or duration is sufficient for detecting the ascending waveform or descending waveform of the respiratory airflow RA.

The processing unit 14 is connected with the airflow sensor 12. The processing unit 14 receives a flow variation signal of the respiratory airflow RA and employs an algorithm to analyze the amplitude A of the respiratory airflow RA and measure the time T of the respiratory cycle, thereby obtaining a flow index FI. Moreover, the algorithm determines whether the respiratory airflow RA represents the normal respiration state NRS or the abnormal respiration state ANRS according to the flow index FI.

For example, the algorithm obtains amplitudes A and times T of a plurality of sampling points. The amplitudes A and times T determines an inspiration waveform and an expiration waveform. The algorithm normalizes the amplitudes A and times T of one of the inspiration waveform and the expiration waveform, so as to establish a normalized waveform. The algorithm calculates the differences between a standard waveform and the normalized waveform, or the differences between a linear equation and the normalized waveform, so as to obtain the flow index FI.

In another embodiment, the reference waveform is selected from a plurality of standard waveforms, and the selected standard waveform is most similar to the normalized waveform.

The display unit 16 is connected with the processing unit 14. The display unit 16 displays the normal respiration state NRS or the abnormal respiration state ANRS.

In another embodiment, the respiratory waveform recognition system 10 further comprises a storage unit (not shown) connected with the processing unit 12 to store the flow index FI.

While at least one exemplary embodiment has been presented in the foregoing detailed description, it should be appreciated that a vast number of variations exist. It should also be appreciated that the exemplary one or more embodiments described herein are not intended to limit the scope, applicability, or configuration of the claimed subject matter in any way. Various changes can be made in the function and arrangement of elements without departing from the scope defined by the claims, which include known equivalents and foreseeable equivalents at the time of filing this patent application.

What is claimed is:

1. A respiratory waveform recognition method for identification of a normal respiration state and an abnormal respiration state from a waveform of a respiratory airflow, the respiratory waveform recognition method comprising:
detecting the respiratory airflow in a respiratory cycle;
measuring an amplitude of the respiratory airflow and a duration of the respiratory cycle, wherein the duration is sufficient for detecting an ascending waveform or a descending waveform of the respiratory airflow;
using a plurality of sampling points to determine an inspiration waveform and an expiration waveform according to the amplitude and the duration;
normalizing the amplitude and the duration of one of the inspiration waveform and the expiration waveform, so as to establish a normalized waveform; and
accumulating the differences between the normalized waveform and a reference waveform to calculate a flow index useful for the identification of the normal respiration state and the abnormal respiration state.

2. The respiratory waveform recognition method of claim 1, wherein the reference waveform is in compliance with a first-order linear equation C1(i) of the following mathematical representation:
α₁·i+α₂; and
the flow index has the following mathematical representation:
Σᵢ₌ₓ^{y}|C1(i)−F(i)|;
wherein α₁ and α₂ are constants, x is a starting point of the sampling points, y is an ending point of the sampling points, i is each sampling point between x and y, and F(i) is the amplitude of the normalized waveform at the sampling point i.

3. The respiratory waveform recognition method of claim 1, wherein the reference waveform is in compliance with a second-order linear equation C2 (i) of the following mathematical representation:
α₁·i²+α₂·i+α₃; and
the flow index has the following mathematical representation:
Σᵢ₌ₓ^{y}|C2(i)−F(i)|;
wherein α₁, α₂ and α₃ are constants, x is a starting point of the sampling points, y is an ending point of the sampling points, i is each sampling point between x and y, and F(i) is the amplitude of the normalized waveform at the sampling point i.

4. The respiratory waveform recognition method of claim 1, wherein the reference waveform is in compliance with a third-order linear equation C3(i) of the following mathematical representation:
α₁·i³+α₂·i²+α₃·i+α₄; and
the flow index has the following mathematical representation:
Σᵢ₌ₓ^{y}|C3(i)−F(i)|;
wherein α₁, α₂, α₃ and α₄ are constants, x is a starting point of the sampling points, y is an ending point of the sampling points, i is each sampling point between x and y, and F(i) is the amplitude of the normalized waveform at the sampling point i.

5. The respiratory waveform recognition method of claim 1, wherein the reference waveform is in compliance with a third-order weighted linear equation C4(i) of the following mathematical representation:
α₁·i³+α₂·i²+α₃·i+α₄; and
the flow index has the following mathematical representation:
$${\sum }_{i = x}^{y} W ( i ) \times \vert C 4 ( i ) - F ( i ) \vert ;$$
$$wherein$$
$$W ( i ) \{ \begin{matrix}A , & i = x , y \\ B , & i = MAX ( F ( i ) ) \\ C , & others\end{matrix} ;$$
wherein α₁, α₂, α₃ and α₄ are constants, x is a starting point of the sampling points, y is an ending point of the sampling points, i is each sampling point between x and y, W(i) is a weighting function, A, B and C individually represent a weighting factor, and F(i) is the amplitude of the normalized waveform at the sampling point i.

6. The respiratory waveform recognition method of claim 5, wherein the weighting factor A ranges from 50 to 100, the weighting factor B ranges from 200 to 400, and the weighting factor C is 1.

7. The respiratory waveform recognition method of claim 5, further comprising:
setting a plurality of flow thresholds; and
comparing the flow index with the flow thresholds to determine obstruction severity of an airway.

8. The respiratory waveform recognition method of claim 7, wherein, according to the respiratory airflow of the airway, the obstruction severity is categorized into the following types: clear airway, mild obstruction, severe obstruction and snoring.

9. The respiratory waveform recognition method of claim 1, wherein the reference waveform is in compliance with one of a plurality of standard waveforms S(i), and the flow index has the following mathematical representation:
$$\sqrt{\frac{{\sum }_{i = x}^{y} {( Ki - μ )}^{2}}{L}} ;$$
$$wherein$$
$$Ki = {\sum }_{i = x}^{y} R \times \vert F ( i ) - S ( i ) \vert ;$$
$$μ = \frac{1}{L} \sum Ki ;$$
$$R = \frac{\sum S ( i )}{\sum F ( i )} ;$$
wherein x is a starting point of the sampling points, y is an ending point of the sampling points, i is each sampling point between x and y, L is the duration between the starting point and the ending point, Ki is an absolute error between any one of the standard waveforms and the normalized waveform, μ is the mean of absolute errors, R is a ratio of area under curve of any one of the standard waveforms and the normalized waveform, and F(i) is the amplitude of the normalized waveform at the sampling point i.

10. The respiratory waveform recognition method of claim 9, wherein the starting point is set to 20% of the sampling points and the ending point is set to 80% of the sampling points.

11. A respiratory waveform recognition method for identification of a normal respiration state and an abnormal respiration state from a respiratory airflow, the respiratory waveform recognition method comprising:
detecting the respiratory airflow in a respiratory cycle;
measuring an amplitude of the respiratory airflow and a duration of the respiratory cycle, wherein the duration is sufficient for detecting an ascending waveform or a descending waveform of the respiratory airflow;
using a plurality of sampling points to determine an inspiration waveform and an expiration waveform according to the amplitude and the duration;
normalizing the amplitude and the duration of one of the inspiration waveform and the expiration waveform, so as to establish a normalized waveform;
fitting the normalized waveform to a linear equation; and
using a weighted least-squares method to calculate the differences between the normalized waveform and the linear equation, and calculating a flow index from the differences with a weighting function, the flow index being useful for the identification of the normal respiration state and the abnormal respiration state.

12. A respiratory waveform recognition method for identification of a normal respiration state and an abnormal respiration state from a respiratory airflow, the respiratory waveform recognition method comprising:
detecting the respiratory airflow in a respiratory cycle;
measuring an amplitude of the respiratory airflow and a duration of the respiratory cycle, wherein the duration is sufficient for detecting an ascending waveform or a descending waveform of the respiratory airflow;
using a plurality of sampling points to determine an inspiration waveform and an expiration waveform according to the amplitude and the duration;
normalizing the amplitude and the duration of one of the inspiration waveform and the expiration waveform, so as to establish a normalized waveform;
comparing a plurality of standard waveforms with the normalized waveform, so as to set one of the standard waveforms as a reference waveform which is most similar to the normalized waveform among the standard waveforms; and
accumulating the differences between the normalized waveform and the reference waveform to calculate a flow index useful for the identification of the normal respiration state and the abnormal respiration state.

13. The respiratory waveform recognition method of claim 12, wherein the differences represent at least one of a least absolute error value or a least square error value.

14. The respiratory waveform recognition method of claim 12, wherein the abnormal respiration state indicates apnea, hypopnea or flow limitation.

15. A respiratory waveform recognition system for identification of a normal respiration state and an abnormal respiration state from a respiratory airflow, the respiratory waveform recognition system comprising:
an airflow sensor for detecting flow variation of the respiratory airflow for a duration, wherein the duration is sufficient for detecting an ascending waveform or a descending waveform of the respiratory airflow;
a processing unit connected to the airflow sensor, the processing unit receiving a signal corresponding to the flow variation of the respiratory airflow, using an algorithm to analyze an amplitude of the respiratory airflow and measuring the duration to calculate a flow index, the algorithm being adapted to determine, according to the flow index, whether the respiratory airflow represents the normal respiration state or the abnormal respiration state; and
a display unit connected to the processing unit, the display unit displaying the normal respiration state or the abnormal respiration state;
wherein the algorithm samples the amplitude and the duration of a plurality of sampling points, the amplitude and the duration determine an inspiration waveform and an expiration waveform, and the algorithm normalizes the amplitude and the duration of one of the inspiration waveform and the expiration waveform, so as to establish a normalized waveform, the algorithm obtaining the flow index by calculating the differences between a reference waveform and the normalized waveform, or by calculating the differences between a linear equation and the normalized waveform.

16. The respiratory waveform recognition system of claim 15, further comprising a storage unit connected to the processing unit for storing the flow index.

17. The respiratory waveform recognition system of claim 15, wherein the reference waveform is selected from a plurality of standard waveforms and is most similar to the normalized waveform among the standard waveforms.
